(19) United States
(12) Patent Application Publication
(10) Pub. No.: US 2025/0160808 A1
(43) Pub. Date: May 22, 2025

(54) LAPAROSCOPY SYSTEMS, LAPAROSCOPY POINT OF ACCESS AND REMOVAL DEVICES, AND METHODS OF USE

(71) Applicant: Antonio Dominguez-Palomar, Dubai (AE)

(72) Inventor: Antonio Dominguez-Palomar, Dubai (AE)

(21) Appl. No.: 18/920,064

(22) Filed: Oct. 18, 2024

Related U.S. Application Data
(60) Provisional application No. 63/600,824, filed on Nov. 20, 2023.

(51) Int. Cl. A61B 17/00; A61B 17/064; A61B 17/3205

(52) U.S. Cl. CPC A61B 17/00234; A61B 17/0644; A61B 17/32053

(57) ABSTRACT

Laparoscopy point of access and removal devices, laparoscopy systems, and methods of use. Such a laparoscopy point of access and removal device includes a cylindrical sidewall including an interior wall and an exterior wall, and a cap attached to the cylindrical sidewall, enclosing a top portion of the cylindrical sidewall forming an interior cavity of the laparoscopy point of access and removal device. The cap includes a first access port configured to accept a shaft of a linear stapler and a circular stapler into the interior cavity of the laparoscopy point of access and removal device. The laparoscopy point of access and removal device is configured to be attached to a wound protector-retractor for use during laparoscopic surgery.

### BACKGROUND OF THE INVENTION

The present invention generally relates to laparoscopy systems, laparoscopy point of access and removal devices, and methods of their use. The invention particularly relates to utilizing a laparoscopy point of access and removal device during laparoscopy to increase cleanliness and reduce contamination of a surgical site to promote better surgical outcomes.

Laparoscopic surgery is known as a minimally invasive procedure performed in the abdomen or pelvis with the use of a laparoscope to allow viewing within the area of the body undergoing the surgical procedure. While forms of laparoscopic surgery have entailed routing a laparoscope to the surgical area from a location remote to the surgical area, procedures and devices have been developed in which the laparoscope can be inserted directly through a surgical incision within the surgical area. As a nonlimiting example, surgical devices such as a wound protector-retractor and access platform are commercially available from Applied Medical Resources Corporation under the names ALEXIS® and GelPOINT®, respectively. The ALEXIS® device has been described as providing circumferential retraction to facilitate visualization of the margins of a surgical incision. The GelPOINT® device has been described as used in combination with the ALEXIS device to create a pseudoabdomen platform capable of facilitating the triangulation of laparoscopic instruments.

Currently-available wound protector-retractor and access platforms are constructed entirely of flexible, gel-like materials with no or limited structural support. Further, these available wound protector-retractor and access platforms lack designated access ports capable of supporting a broad range of instruments and procedures. Therefore, there exists a need for laparoscopy systems that are more versatile while offering safe and clean medical procedures.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, laparoscopy systems and laparoscopy point of access and removal devices capable of use during laparoscopic surgery in combination with a wound protector-retractor.

According to a nonlimiting aspect of the invention, a laparoscopy point of access and removal device includes a rigid sidewall having a cylindrical shape and a rigid cap contiguous with an upper extent of the sidewall. Together the sidewall and the cap define an interior cavity exposed at a lower side of the device. A first access port protrudes from an upper surface of the cap. The first access port has a passage configured to accept a shaft of a circular stapler into the interior cavity of the device, and further has a valve disposed in the passage thereof. At least a second access port protrudes from a semi-rigid or flexible portion of the cap. The second access port has a passage configured to accept shafts of smaller medical devices, such as a trocar or a linear stapler into the interior cavity of the device.

According to another nonlimiting aspect of the invention, a laparoscopy system configured to assist in laparoscopic surgery includes a wound protector-retractor including an upper ring having an outer sidewall, a lower ring axially spaced apart from the upper ring, and a film extending between and connecting the upper and lower rings. A laparoscopy point of access and removal device as described above is coupled to the upper ring of the wound protector-retractor, and the sidewall of the laparoscopy point of access and removal device is connected to outer sidewall of the upper ring to form a substantially airtight seal, which may be achieved by the use of a clamp.

According to still another nonlimiting aspect of the invention, a method of performing laparoscopic surgery with a laparoscopy system includes inserting a lower ring of a wound protector-retractor through an incision of a patient to cause a film of the wound protector-retractor to reside against a wall of the incision, folding an upper ring of the wound protector-retractor down over the film until the upper ring is in contact with an epidermis of the patient adjacent the incision of the patient, attaching a sidewall of a laparoscopy point of access and removal device to an outer sidewall of the upper ring to form a substantially airtight seal, inserting a shaft of a surgical stapler into and through a passage of a first access port located on a cap of the laparoscopy point of access and removal device and into the incision, stapling an incision with the surgical stapler, removing the surgical stapler from the incision and through the passage of the access port, and continuing with the laparoscopic surgery. Optionally, the method may further include an extraction step, in which the laparoscopy point of access and removal device is removed from the wound protector-retractor and tissue is extracted through the wound protector-retractor, after which the laparoscopy point of access and removal device can be reattached before continuing with the laparoscopic surgery.

Technical aspects of devices and methods as described above preferably include the capability of performing laparoscopic surgery with a broad range of instruments and procedures while promoting safety and cleanliness and reducing contamination of an incision site. Additionally, when mounted to a wound protector-retractor, the laparoscopy point of access and removal device allows for a consistent air pressure of the incision site and may reduce the number of sutures needed. Lastly, consistent surgical results are more readily reproducible with use of the point of access and removal device.

These and other aspects, arrangements, features, and/or technical effects will become apparent from the following detailed description as well as any drawings.

### BRIEF DESCRIPTION OF THE SEVERAL VIEWS OF THE FIGURES

FIG. 1 schematically represents a side view of a laparoscopy point of access and removal device in accordance with a nonlimiting embodiment of the invention.

FIG. 2 schematically represents a top view of the laparoscopy point of access and removal device of FIG. 1.

FIG. 3 schematically represents a top view of an alternative embodiment of a laparoscopy point of access and removal device in accordance with a nonlimiting embodiment of the invention.

FIG. 4 schematically represents a top view of the laparoscopy point of access and removal device of FIG. 3.

FIG. 5 schematically represents a side view of a flexible portion of the laparoscopy point of access and removal device of FIG. 3.

FIG. 6 schematically represents a side view of a laparoscopy incision wound protector-retractor of the prior art.

FIG. 7 schematically represents a side view of the laparoscopy point of access and removal device of FIG. 3 mounted to the wound protector-retractor of FIG. 6 and installed in an incision of a patient.

FIG. 8 schematically represents a side view of the laparoscopy point of access and removal device of FIG. 1 mounted to the wound protector-retractor of FIG. 6.

FIG. 9 schematically represents a side view of the laparoscopy point of access and removal device of FIG. 3 mounted to the wound protector-retractor of FIG. 6.

### DETAILED DESCRIPTION OF THE INVENTION

The intended purpose of the following detailed description of the invention and the phraseology and terminology employed therein is to describe what is shown in the drawings, which include the depiction of and/or relate to one or more nonlimiting embodiments of the invention, and to describe certain but not all aspects of what is depicted in the drawings. The following detailed description also identifies certain but not all alternatives of the embodiment(s) depicted in the drawings. As nonlimiting examples, the invention encompasses additional or alternative embodiments in which one or more features or aspects shown and/or described as part of a particular embodiment could be eliminated, and also encompasses additional or alternative embodiments that combine two or more features or aspects shown and/or described as part of different embodiments.

Therefore, the appended claims, and not the detailed description, are intended to recite what at least provisionally are believed to be aspects of the invention, including certain but not necessarily all of the aspects and alternatives described in the detailed description.

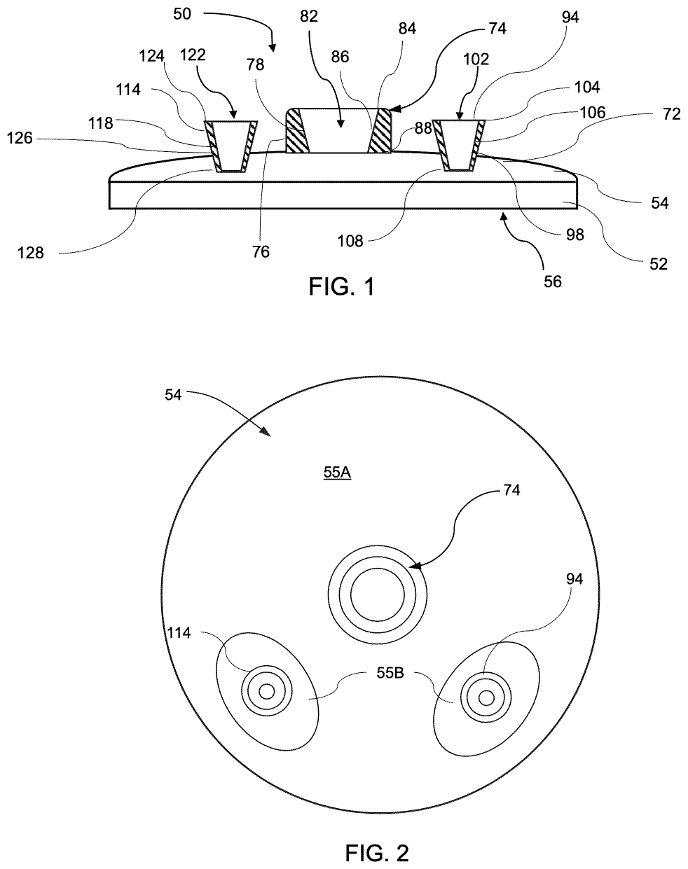

The following disclosure describes various aspects of nonlimiting embodiments of laparoscopy point of access and removal devices (hereinafter sometimes simply referred to as a “device” or “devices” as a matter of convenience) and components thereof that are represented in FIG. 1 through 4.

To facilitate the description provided below of the embodiments represented in the drawings, relative terms, including but not limited to, “proximal,” “distal,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to an orientation of the point of access and removal devices during their use while positioned on a patient undergoing laparoscopic surgery. All such relative terms are intended to indicate the construction and relative orientations of components and features of the devices and therefore are relative terms that are useful to describe the illustrated embodiments and indicate the construction, installation, and use of the devices, and in doing so may help to define the scope of the invention.

FIGS. 1 and 2 schematically represent a first nonlimiting embodiment of a laparoscopy point of access and removal device 50. The device 50 includes a sidewall 52 having a cylindrical shape and a cap 54 contiguous with an upper extent of the sidewall 52. Together, the sidewall 52 and cap 54 define an interior cavity 56 exposed at the lower side (not visible) of the device 50.

The cap 54 has a dome-like configuration and is preferably constructed from combinations of rigid materials and non-rigid or semi-rigid materials having self-sealing characteristics. In the nonlimiting embodiment represented in FIGS. 1 and 2, the cap 54 has at least one rigid portion 55A formed of a relatively rigid material and two relatively non-rigid or two semi-rigid portions 55B that are preferably self-sealing (and for convenience are hereinafter referred to as self-sealing portions 55B) that are integrally incorporated into the cap 54. It is also contemplated that the rigid and self-sealing portions 55A and 55B may be separate portions assembled within or attached to the cap 54. The rigid material of the rigid portion 55A may be formed of a medical-grade material suitable for use in a surgical procedure, a nonlimiting example of which is KRATON®, and the two self-sealing portions 55B are preferably, though not necessarily, formed of an elastomeric gel having self-sealing characteristics, nonlimiting examples of which include materials that contain a combination of KRATON® and an oil. Particular examples of these materials are described in U.S. Pat. No. 7,473,221, which is incorporated herein by reference. The self-sealing portions 55B are configured to be capable of being penetrated by a medical instrument, as a nonlimiting example, a trocar.

Access ports 74, 94, and 114 are shown as protruding from an upper surface 72 of the cap 54. A first access port 74 is supported by the rigid portion 55A of the cap 54, whereas the second and third access ports 94 and 114 are supported by the self-sealing portions 55B. The access port 74 has a cylindrical shape, with an upper portion 84 and a lower portion 88. The lower portion 88 is rigidly connected and supported by the rigid portion 55A of the cap 54 and preferably does not move or otherwise deform independent of the cap 54. The rigid portion 55A of the cap 54 and the first access port 74 connected thereto, allow for the first access port 74 to act as a fulcrum during surgery. For example, in addition to providing an insertion point for a circular stapler, the first access portion 74 can provide support for a trocar or act as pivot point for the trocar, such as when the trocar is used in combination with a linear (straight) stapler. Conversely, the self-sealing portions 55B are capable of being elastically distorted independent of the cap 54 to allow for some limited movement. The first access port 74 includes an internal wall 78 surrounding and defining an internal passage 82 that extends from the exterior of the device 50, through the cap 54, and into the interior cavity 56 of the device 50. The internal passage 82 includes a cylindrical funnel shape, with the diameter at the upper portion 84 being greater than the diameter at the lower portion 88. The upper portion 84, the lower portion 88, and a middle portion 86 therebetween define three distinct sections of the port 74. The upper and lower portions 84 and 88 are preferably formed of one or more semi-rigid materials to allow for a medical instrument to be inserted into the passage 82. The middle portion 86 is preferably formed of a material that is sufficiently more rigid than the materials of the upper and lower portions 84 and 88 to assist in stabilizing a medical instrument inserted into or through the passage 82. The lower portion 88 may further comprise an embedded flexible material within the passage 82 to create a fluidic seal between the first access port 74 and a medical instrument inserted through the passage 82. The configuration of the first access port 74 is such that it allows for both linear staplers (e.g., about 12 millimeters in diameter) and circular staplers (e.g., about 15-33 millimeters in diameter) to be inserted through the passage 82. The first access port 74 preferably also includes a valve (not shown) within the passage 82 for use in connection with a circular stapler for insertion and manipulation.

The second and third access ports 94 and 114 have a generally cylindrical funnel shape and are particularly adapted for accepting a trocar and/or linear stapler (e.g., about 12 millimeters in diameter). Similar to the first access port 74, each of the second and third access ports 94 and 114 has, respectively, an upper portion 104 and 124 having a greater outer diameter than a lower portion 108 and 128 thereof, and an internal wall 98 and 118 surrounding and defining an internal passage 102 and 122 that extends from the exterior of the device 50, through the cap 54, and into the interior cavity 56 of the device 50. Also similar to the first access portion 74, the second and third access ports 94 and 114 may have three distinct sections formed by their respective upper portions 104 and 124, lower portions 108 and 128, and middle portions 106 and 126 therebetween, in which their middle portions 106 and 126 are formed of a material that is more rigid than the materials of the upper and lower portions 104, 108, 124, and 128. The configurations of the second and third access ports 94 and 114 are preferably such that a trocar may be inserted into their passages 102 and 122, respectively, and then into the interior cavity 56 of the device 50 by puncturing their respective self-sealing portions 55B, allowing for the placement of the trocar and a linear stapler through the trocar. It is contemplated that either or both of the passages 102 and 122 may be configured with a valve integrated within, or alternatively the passages 102 and 122 may be configured to accept a valve along with a trocar, to avoid the need to puncture the self-sealing portion 55B.

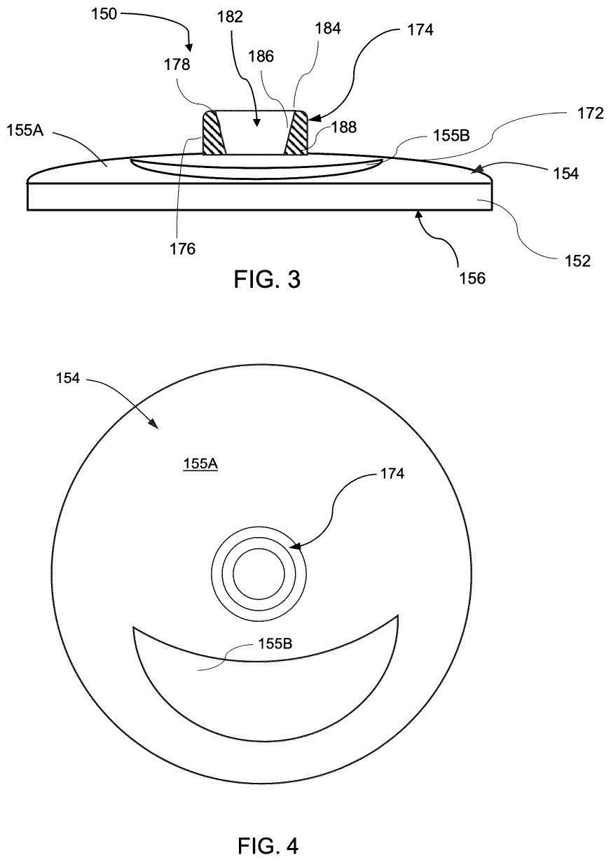

FIGS. 3 and 4 schematically represent an alternative nonlimiting embodiment of a laparoscopy point of access and removal device 150. The device 150 includes a sidewall 152 having a cylindrical shape and a cap 154 contiguous with an upper extent of the sidewall 152. Together, the sidewall 152 and cap 154 define an interior cavity 156 exposed at the lower side (not visible) of the device 150. Similar to the embodiment of FIGS. 1 and 2, the cap 154 has a dome-like configuration and is preferably constructed from combinations of rigid materials and non-rigid or semi-rigid materials having self-sealing characteristics. In the nonlimiting embodiment represented in FIGS. 3 and 4, the cap 154 has a rigid portion 155A formed of a relatively rigid material and a relatively non-rigid or two semi-rigid portion 155B that is preferably self-sealing (and for convenience are hereinafter referred to as a self-sealing portion 155B) and configured to be penetrated by a medical instrument such as a trocar. It is also contemplated that there may be multiple self-sealing portions, and that they may be separate portions attached to the cap 154.

A first access port 174 protrudes from an upper surface 172 of the cap 154 and is supported by the rigid portion 155A of the cap 154. The first access port 174 has a cylindrical shape, with an upper portion 184 and a lower portion 188. The lower portion 188 is rigidly connected and supported by the rigid portion 155A of the cap 154 and may not move or otherwise deform independent of the cap 164. The rigid portion 155A of the cap 154 and the first access port 174 connected thereto, allow for the first access port 174 to act as a fulcrum during surgery, providing support for a trocar or acting as pivot point for the trocar. The first access port 174 includes an internal wall 178 surrounding and defining an internal passage 82 that extends from the exterior of the device 150, through the cap 154, and into the interior cavity 156 of the device 150. The internal passage 182 includes a cylindrical funnel shape, with the diameter at the upper portion 184 being greater than the diameter at the lower portion 188. The upper portion 184, the lower portion 188, and a middle portion 186 therebetween define three distinct sections of the port 174. The upper and lower portions 184 and 188 are preferably formed of one or more semi-rigid materials to allow for a medical instrument to be inserted into the passage 182. The middle portion 186 is preferably formed of a material that is sufficiently more rigid than the materials of the upper and lower portions 184 and 188 to assist in stabilizing a medical instrument inserted into or through the passage 182. The lower portion 188 may further comprise an embedded flexible material within the passage 182 to create a fluidic seal between the first access port 174 and a medical instrument inserted through the passage 182.

The configuration of the first access port 174 can be generally as described for the first access port 74 of FIGS. 1 and 2. The first access port 174 preferably allows for both circular staplers (e.g., about 15-33 millimeters in diameter) and trocars (e.g., about 12 millimeters in diameter) to be inserted through its passage 182. The first access port 174 preferably also includes a valve (not shown) within the passage 182 for use in connection with a circular stapler for insertion and manipulation.

The self-sealing portion 155B of the cap 154 is adapted for customized insertion of trocars or existing materials and procedures as depicted in FIG. 5. FIG. 5 schematically represents a close up perspective of a portion of the device 150 of FIG. 3 with a trocar port 200 inserted through the self-sealing portion 155B and into the interior cavity 156 of the cap 154. The self-sealing portion 155B can be punctured for insertion of the trocar 200 using various known devices, as a nonlimiting example, an obturator disclosed in U.S. Pat. No. 8,790,308.

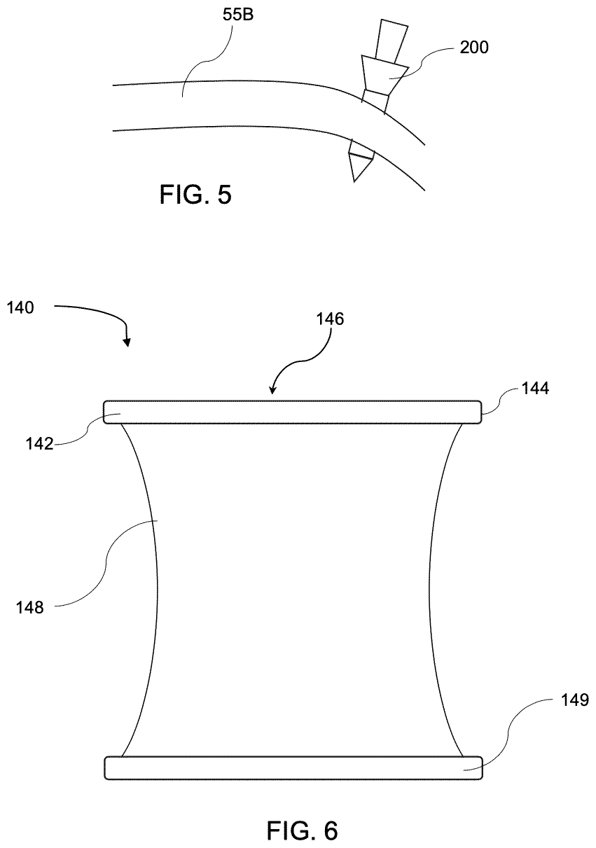

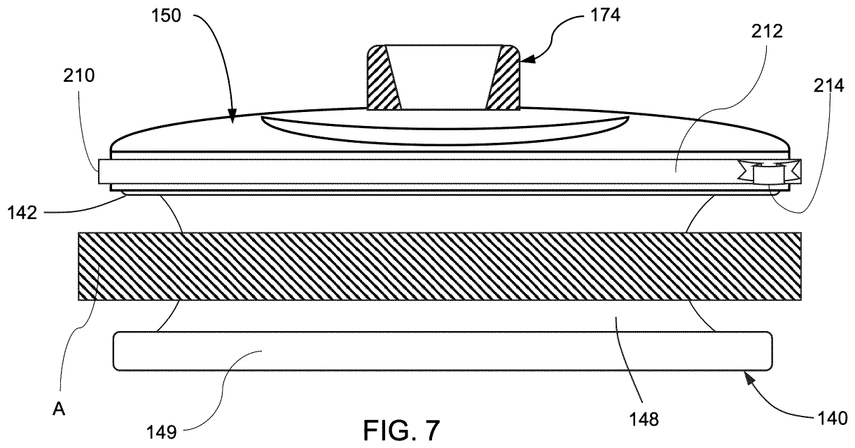

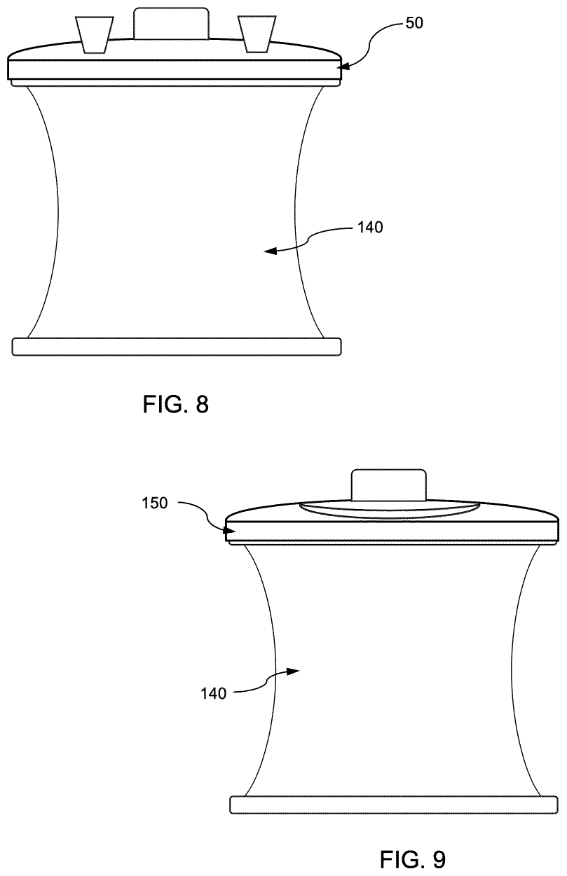

FIG. 6 schematically represents a laparoscopy incision wound protector-retractor (WPR) 140, as a nonlimiting example, the ALEXIS® wound protector-retractor commercially available from Applied Medical Resources Corporation. The WPR 140 is a device commonly used within laparoscopy procedures, as disclosed in U.S. Pat. No. 7,650,887. The WPR 140 includes an upper ring 142 having an outer sidewall 144, a lower ring 149 axially spaced apart from the upper ring 142, and a film 148 connecting the upper and lower rings 142 and 149. Together, the rings 142 and film 148 define an interior passage 146 that is continuous through the entire WPR 140. A film 148 is connected to the upper ring 142 and extends away from the upper ring 142. The point of access and removal devices 50 and 150 depicted in FIGS. 1 through 4 are configured to be utilized with the WPR 140 during laparoscopic surgery, and in particular to be secured to the upper ring 142 so that the sidewall 52 and 152 of each device 50 and 150 is capable of surrounding and coupling with the upper ring 142 as depicted in FIGS. 7 through 9.

FIG. 7 schematically represents the device 150 of FIG. 3 placed on the WPR 140 that has been placed in an incision A in a patient The device 150 includes a clamp 210, which in the nonlimiting example shown is configured as a band 212 to extend around the circumference of the device 50 and 150 sidewall 52 and 152 to secure the device 50 and 150 to the WPR 140 with a buckle 214. The clamp 210 enables the devices 50 and 150 to more easily attach, remove, and re-attach to the WPR 140 during surgery. Further, the clamp 210 results in a rigid, compact, and airtight connection between the WPR 140 and the device 50 and 150, thereby giving support to the device 50 and 150 as well as the access ports 74, 94, 114 and 174.

Utilizing the devices 50 and 150 in combination with the WPR 140 during a surgical procedure allows for added, safety, cleanliness, and reduced contamination of the incision, and additionally normalizes air pressure changes and may reduce the number of sutures needed. When performing laparoscopic surgery, a surgeon or other healthcare professional inserts the lower ring 149 of the WPR 140 through the surgical incision A in a patient. The film 148 of the WPR 140 is caused to reside against a wall of the incision. The upper ring 142 of the WPR 140 is folded or rolled down over the film 148 until the upper ring 142 is in contact with the epidermis of the patient, adjacent to the incision of the patient. The sidewall 52 of the device 50 is then attached to the outer sidewall 144 of the upper ring 142 to form a substantially fluid-tight and preferably airtight seal. The first access port 174 is adapted to accept a circular stapler (not shown), whose shaft (e.g., about 15-33 millimeters in diameter) is inserted into and through the passage 82 of the first access port 74 and subsequently through the incision. Tissue can then be stapled with stapler, after which the stapler is removed from the incision and the access port 74, and the surgery continues. Optionally, the procedure may further include an extraction step, in which the device 50 is removed from the WPR 140 and tissue is extracted through the interior passage 146 of the WPR 140, after which the device 50 can be reattached to the WPR 140 before continuing with the laparoscopic surgery.

As previously noted above, though the foregoing detailed description describes certain aspects of one or more particular embodiments of the invention, alternatives could be adopted by one skilled in the art. For example, the laparoscopy point of access and removal device and its components could differ in appearance from the embodiments described herein and shown in the drawings, and functions of certain components of the laparoscopy point of access and removal device could be performed by components of different construction but capable of a similar (though not necessarily equivalent) function. As such, and again as was previously noted, it should be understood that the invention is not necessarily limited to any particular embodiment described herein or illustrated in the drawings.

1. A laparoscopy point of access and removal device configured to be attached to a wound-retractor protector, the laparoscopy point of access and removal device comprising:
a rigid sidewall having a cylindrical shape;
a rigid cap contiguous with an upper extent of the sidewall, together the sidewall and the cap defining an interior cavity exposed at a lower side of the device;
at least a first self-sealing portion disposed within the cap; and
a first access port protruding from an upper surface of the cap, the first access port having a passage configured to accept a shaft of a circular stapler into the interior cavity of the device, the first access port having a valve disposed in the passage thereof.

2. The laparoscopy point of access and removal device of claim 1, further comprising at least a second access port protruding from the first self-sealing portion of the cap, the second access port having a passage configured to accept a shaft of a trocar or a linear stapler into the interior cavity of the device, wherein the self-sealing portion of the cap is semi-rigid, capable of being elastically distorted independent of the cap.

3. The laparoscopy point of access and removal device of claim 2, further comprising a second self-sealing portion disposed within the cap and a third access port protruding from the second self-sealing portion of the cap, the third access port having a passage configured to accept a shaft of a trocar or a linear stapler into the interior cavity of the laparoscopy point of access and removal device.

4. The laparoscopy point of access and removal device of claim 3, wherein at least one of the second and third access ports comprises a valve disposed in the passage thereof.

5. The laparoscopy point of access and removal device of claim 3, wherein at least one of the second and third access ports does not have a valve disposed in the passage thereof.

6. The laparoscopy point of access and removal device of claim 1, wherein the self-sealing portion of the cap is flexible and capable of being penetrated by a medical instrument.

7. A laparoscopy system configured to assist in laparoscopic surgery, the laparoscopy system comprising:
a wound protector-retractor including an upper ring having an outer sidewall, a lower ring axially spaced apart from the upper ring, and a film extending between and connect the upper and lower rings;
the laparoscopy point of access and removal device of claim 1 coupled to the upper ring of the wound protector-retractor, and
a clamp disposed around the circumference of the sidewall of the laparoscopy point of access and removal device and connecting the laparoscopy point of access and removal device to the outer sidewall of the upper ring to form a substantially airtight seal.

8. A method of performing laparoscopic surgery with a laparoscopy system, the method comprising:
inserting a lower ring of a wound protector-retractor through an incision of a patient to cause a film of the wound protector-retractor to reside against a wall of the incision;
folding an upper ring of the wound protector-retractor down over the film until the upper ring is in contact with an epidermis of the patient adjacent the incision of the patient;
attaching a sidewall of a laparoscopy point of access and removal device to an outer sidewall of the upper ring to form a substantially airtight seal;
inserting a shaft of a surgical stapler into and through a first access port located on a rigid cap of the laparoscopy point of access and removal device and into the incision;
stapling an incision with the surgical stapler;
removing the surgical stapler from the incision through the access port; and continuing with the surgery.

9. The method of claim 8, wherein the surgical stapler is a circular stapler.

10. The method of claim 8, further comprising:
removing the laparoscopy point of access and removal device from the wound protector-retractor;
extracting tissue through the wound protector-retractor; and then
reattaching the laparoscopy point of access and removal device to the wound protector-retractor before continuing with the laparoscopic surgery.
